(19) United States
(12) Patent Application Publication
(10) Pub. No.: US 2018/0067177 A1
(43) Pub. Date: Mar. 8, 2018

(54) METHOD FOR ESTABLISHING IMPLANT INFORMATION ABOUT A PATIENT FROM A MAGNETIC RESONANCE SCAN

(71) Applicant: Siemens Healthcare GmbH, Erlangen (DE)

(72) Inventors: Klaus LUDWIG, Erlangen (DE); Stefan SCHOR, Erlangen (DE); Dani SIKIRIC, Erlangen (DE)

(73) Assignee: Siemens Healthcare GmbH, Erlangen (DE)

(21) Appl. No.: 15/695,187

(22) Filed: Sep. 5, 2017

(30) Foreign Application Priority Data
Sep. 5, 2016 (DE) 102016216729.4

(51) Int. Cl. G01R 33/28; A61B 5/055; G01R 33/56

(52) U.S. Cl. CPC G01R 33/288; G01R 33/5608; A61B 5/055

(57) ABSTRACT

In a method and magnetic resonance (MR) apparatus for establishing implant information about a patient based on an MR scan of the patient, the MR scan is performed by implant-safe settings in the control parameters. The MR signals acquired in this scan are reconstructed to produce image data, and the image data are searched for at least one weak-signal region in a computer search. Implant information about the patient are generated by the computer dependent on the result of the search for the at least one weak-signal region, and the implant information is made available as an electronic output from the computer.

### BACKGROUND OF THE INVENTION

### Field of the Invention

The present invention concerns a method for establishing implant information about a patient by implementing a magnetic resonance scan with a magnetic resonance apparatus. The present invention further concerns a magnetic resonance apparatus configured to implement such a method, and a computer-readable data storage medium, containing program code that cause a computer to operate a magnetic resonance apparatus in order to implement such a method.

### Description of the Prior Art

Before performing a magnetic resonance examination of a patient, for safety reasons it is necessary to obtain information about implants that may be present in the patient. To this end, patients are generally asked whether they have an implant. The questioning is generally performed manually by the medical operator (clinician) responsible for carrying out the magnetic resonance examination, and it is possible that the operator may forget to do so, so no information will be available as to possible implants in the patient. It may also occur that patients unwittingly or inadvertently provide incorrect information in this regard, or are even unable to provide any information due to their state of health.

If no information is provided relating to possible implants in the patient or if information is not correct, heretofore it has not been possible properly to take account of implants in the patient for the upcoming magnetic resonance scan. However, this can result in damage, such as burning, to the implant or patient during the magnetic resonance scan.

### SUMMARY OF THE INVENTION

An object of the present invention is to provide implant information about a patient independently of the patient's state.

The method according to the invention for establishing implant information about a patient based on a magnetic resonance scan with a magnetic resonance apparatus, has the following steps:

A magnetic resonance scan is performed so as to acquire magnetic resonance (MR signals from a subject, with the magnetic resonance scan governed by implant-safe settings in the control parameters used to operate the MR scanner. Magnetic resonance image data are reconstructed from the MR signals, and the magnetic resonance image data are searched for at least one weak-signal region. Implant information about the patient is generated in the computer dependent on the result of the search for the at least one weak-signal region, and the implant information is made available from the computer as an electronic output.

In this context, implant information about a patient means information as to whether a patient has an implant or is free of implants. This information is preferably obtained before a medical and/or diagnostic magnetic resonance examination is performed on the patient, so that settings and/or parameter adjustments for the upcoming medical and/or diagnostic magnetic resonance examination can be adapted to the implant information on the patient.

In patients with implants, a medical and/or diagnostic magnetic resonance examination with settings in the control parameters that are based on the assumption that the patient does not have an implant can lead to unwanted heating of the implant and hence to injuries to the patient, in particular burns to the patient. It is therefore necessary to obtain the implant information before the medical and/or diagnostic magnetic resonance examination on the patient. Since this implant information is often not available because, for example, it is not possible to speak to the patient because of the patient's state of health or a medical operator forgot to ask for the appropriate information, the implant information is advantageously acquired or established automatically by means of the magnetic resonance apparatus. To this end, the magnetic resonance apparatus has a control computer containing appropriate software and/or computer programs that, when executed on a core processor unit of the control computer, control the method for establishing implant information on the patient based on the magnetic resonance scan with the magnetic resonance apparatus.

When implementing the method for establishing the implant information about the patient, it is preferable to execute a magnetic resonance scan that is designed as a safety magnetic resonance scan. The magnetic resonance scan, in particular the safety magnetic resonance scan, for establishing the implant information is governed by implant-safe settings in the control parameters. Implant-safe settings should in particular be understood to be settings configured in order to avoid heating of possible implants in the patient during the magnetic resonance scan, in particular the safety magnetic resonance scan, as far as possible. Preferably the implant-safe settings are configured such that safe settings are available for all possible and/or conceivable embodiments of implants so that the possibility of injury to the patient during the magnetic resonance scan, in particular the safety magnetic resonance scan, can be excluded. The implant-safe settings in the control parameters can include, for example, a soft slew rate in the gradient pulses of the magnetic resonance scan, in particular the safety magnetic resonance scan, and/or a maximum SAR value of 1.5 W/kg. In addition, further, implant-safe settings in the control parameters that appear advisable to those skilled in the art are possible.

To use the magnetic resonance apparatus for the magnetic resonance scan, the magnetic resonance apparatus and/or components of the magnetic resonance apparatus are typically controlled, wherein different control parameters can be selected depending on the magnetic resonance apparatus. The selected control parameters can depend upon the magnetic resonance apparatus, the object to be examined and/or the clinical issue. Control parameters can be physical variables such as a voltage to be applied to a component, for example to a radio-frequency coil. A control parameter can also be a duration between two radio-frequency pulses and/or magnetic field gradients to be played out. Control parameters can characterize the magnetic resonance image data to be recorded by, for example, specifying a resolution and/or a signal-noise ratio.

When the magnetic resonance apparatus is controlled, the control parameters are, for example, forwarded to the control computer of the magnetic resonance apparatus, wherein the control computer controls at least one component according to the control parameters. If a control parameter is not a physical variable, the control computer preferably transforms the control parameter into at least one physical variable.

The evaluation of the magnetic resonance image data acquired is preferably performed by the control computer of the magnetic resonance apparatus. To this end, the control computer executes an evaluation algorithm. To establish the implant information, the reconstructed magnetic resonance image data are first searched for at least one weak-signal region using the evaluation algorithm of the control computer. The at least one weak-signal region preferably is a region in which only signals with a low strength were emitted during the magnetic resonance scan, or in which no signals at all were emitted during the magnetic resonance scan. Preferably, the at least one weak-signal region is a signal-free region, so that this at least one weak-signal region, when displayed, appears as a black region from which no signal emanates.

The generation of the implant information dependent on the result of the search for the at least one weak-signal region is preferably performed by the control computer of the magnetic resonance apparatus. If the search result does not have a weak-signal region, the control computer generates negative implant information, indicating to the medical operator that the patient does not have an implant. If the search result indicates the presence at least one weak-signal region or multiple weak-signal regions, the control computer generates positive implant information indicating to the medical operator that the patient has at least one implant.

The invention facilitates a simple and reliable, exact, establishment and/or acquisition of the implant information about the patient. In addition, the establishment and/or acquisition of the implant information about the patient is advantageously no longer reliant on active assistance from the patient or a medical operator responsible for the upcoming medical and/or diagnostic magnetic resonance examination. The establishment and/or acquisition of the implant information on the patient can even be performed independently of a patient's state, i.e. even if the patient is no longer conscious or is in a confused state, as may be the case for patients in a state of dementia or patients with impaired consciousness, such as due to the administration of medication.

In addition, the provision of exact implant information can advantageously increase patient safety during a subsequent medical and/or diagnostic magnetic resonance examination. When the implant information indicates that the patient has an implant, it is then possible to adapt the settings and/or control parameters of the subsequent medical and/or diagnostic magnetic resonance scan to the implant present, or even to dispense with a medical and/or diagnostic magnetic resonance scan. This enables malfunctioning of the implant to be reliably avoided.

In the embodiment of the method wherein the at least one weak-signal region is a signal-free region, thus enables a distinction to be made between an implant region and further regions with a low patient signal strength. It is thereby possible to differentiate between the implant region, i.e. a region of the patient with an implant, and for example a lung region depicting the lung tissue of the patient. This also enables simple and reliable assignment of signal-free regions to implant regions of the patient thus enabling highly informative implant information to be achieved. It is also possible to reduce and/or prevent incorrect information within the implant information.

In a further embodiment of the method, the at least one weak-signal region has a minimum size of greater than 0.5 cm in order to be identified as a weak-signal region, in particular a signal-free region. If the weak-signal region has a minimum size of greater than 0.5 cm, it is preferably identified during the evaluation of the magnetic resonance image data by the evaluation algorithm of the control computer. Preferably, the at least one weak-signal region has a size of greater than 1.0 cm in order to be identified as a weak-signal region, in particular a signal-free region. Particularly advantageously, the at least one weak-signal region has a size of greater than 1.5 cm in order to be identified as a weak-signal region, in particular a signal-free region.

This embodiment of the method according to the invention enables efficient differentiation between implant regions and regions with low signal strength in the magnetic resonance image data. This can also enable simple and reliable assignment of weak-signal regions in the image data to an implant.

The region of interest for the magnetic resonance scan can be the entire body of the patient, i.e., the scan is a whole body scan. This also facilitates the acquisition of implant information on anatomical regions of the patient that are not situated directly within the region of interest for the subsequent medical and/or diagnostic magnetic resonance examination.

The implant-safe settings in the control parameters preferably designate a slice thickness of about 0.5 cm. This enables a particularly short scan time for the magnetic resonance scan for the acquisition of the implant information. In addition, this enables stress, radio-frequency radiation, to be minimized for the patient. A slice thickness of about 0.5 cm should be understood to mean a slice thickness of between 0.4 cm and 0.6 cm. The slice thickness may alternatively a value of about 0.8 cm or about 1.0 cm or about 1.2 cm or about 1.4 cm or about 1.6 cm or about 1.8 cm or about 2.0 cm.

In an embodiment of the invention, the implant-safe settings in the control parameters designate a soft slew rate for any gradient pulse that is activated in the MR scan. This enables a particularly weak and/or soft magnetic resonance scan with which heating of possible implants, and hence injuries to the patient, can be at least reduced and/or prevented.

A soft slew rate in the gradient pulse signals is preferably at the most 20 mT/ms. Such a maximum soft slew rate of the gradient pulses is more preferably 15 mT/ms or 10 mT/ms or 8 mT/ms or 5 mT/ms.

Alternatively or additionally, the implant-safe settings in the control parameters can also have a maximum SAR value of 1.5 W/kg. An SAR value is known as meaning the specific absorption rate. The specific absorption rate is a measure of the absorption of electromagnetic fields in the patient. Herein, the SAR value indicates the power that may be absorbed by the patient for each kg of the patient's weight. The maximum SAR value is preferably at most 1.4 W/kg and more preferably 1.3 W/kg or 1.2 W/kg or 1.1 W/kg or 1.0 W/kg or 0.9 W/kg or 0.8 W/kg or 0.7 W/kg or 0.6 W/kg or 0.5 W/kg or 0.4 W/kg or 0.3 W/kg or 0.2 W/kg or 0.1 W/kg. In addition, a radio-frequency pulse signal radiated in the MR scan preferably has a maximum amplitude of 30 μT. To this end, the radio-frequency pulse signals more preferably have a maximum amplitude of 25 μT or 20 μT or 18 μ or 15 μT. This enables a particularly weak and/or soft magnetic resonance scan with which heating of possible implants, and hence injuries to the patient, can be at least reduced and/or prevented. Malfunctions of an implant can also advantageously be prevented.

In a further embodiment of the method, when positive implant information is provided, the subsequent medical and/or diagnostic magnetic resonance scan of a region of interest of the patient is performed in safety mode. Positive implant information means implant information indicating the presence of at least one implant inside the patient. The safety mode has settings in the control parameters that do not result in critical heating and/or unwanted impairment of the implant when the magnetic resonance scan is performed in safety mode. Thus, in the safety mode, the settings in the control parameters take account of the implant information.

This embodiment of the invention increases the safety of the patient for the magnetic resonance scan of the region of interest.

When negative implant information is provided, the subsequent medical and/or diagnostic magnetic resonance examination of a region of interest of the patient is performed in normal mode. Negative implant information means implant information indicating the absence of implants inside the patient. The normal mode uses settings in the control parameters that do not take account of an implant. This can enable the performance of a particularly time-saving magnetic resonance scan of the region of interest on the patient or also a particularly high quality magnetic resonance scan. Such a magnetic resonance scan of the region of interest of the patient is performed only when negative implant information is available.

In an embodiment of the invention, the implant information is displayed. The implant information is preferably displayed at a display unit, a monitor and/or a screen, of the magnetic resonance apparatus. The implant information can also be transferred to a mobile terminal, for example a smartphone or a tablet-PC, belonging to a user, or to a display unit that is separate from the magnetic resonance apparatus, and displayed there. This can be beneficial, for example if the person planning the medical and/or diagnostic magnetic resonance scan of the patient is not present at the site of the magnetic resonance examination.

This embodiment of the invention enables transfer of information to the medical operator responsible for the magnetic resonance scan or a person planning the magnetic resonance scan. This also enables the implant information to be provided during the setting of control parameters for the upcoming medical and/or diagnostic magnetic resonance scan of the region of interest of the patient.

The invention also encompasses a magnetic resonance apparatus having a control computer that operates the scanner of the magnetic resonance apparatus so as to implement the method for establishing implant information about a patient as described above.

The magnetic resonance apparatus according to the invention enables simple and reliable, in particular exact, establishment and/or acquisition of the implant information about the patient. In addition, the establishment and/or acquisition of the implant information about the patient is particularly advantageously no longer reliant on active assistance from the patient or a medical operator responsible for the upcoming medical and/or diagnostic magnetic resonance examination. In particular, the establishment and/or acquisition of the implant information about the patient can also be performed independently of the patient's state, i.e. even if the patient is no longer conscious is or in a confused state, for example, patients in a state of dementia or patients impaired consciousness in particular as a result of the administration of medication.

In addition, the provision of exact implant information increases patient safety during a subsequent medical and/or diagnostic magnetic resonance examination. In particular when implant information indicates that the patient has an implant, it is possible to adapt the settings and/or control parameters of the subsequent medical and/or diagnostic magnetic resonance scan to the implant present or even to dispense with a medical and/or diagnostic magnetic resonance scan.

The advantages of the magnetic resonance apparatus according to the invention substantially correspond to the advantages of the method according to the invention, which are described above in detail. Features, advantages and alternative embodiments of the method apply as well to the apparatus.

The invention also encompasses a non-transitory, computer-readable data storage medium encoded with programming instructions that, when the storage medium is loaded into a control computer or computer system of a magnetic resonance apparatus cause the control computer or computer system to operate the magnetic resonance apparatus in order to implement any or all of the embodiments of the method according to the invention, as described above.

### BRIEF DESCRIPTION OF THE DRAWINGS

FIG. 1 schematically illustrates a magnetic resonance apparatus according to the invention.

FIG. 2 is a flowchart of the method according to the invention for establishing implant information about a patient based on a magnetic resonance scan with a magnetic resonance apparatus.

### DESCRIPTION OF THE PREFERRED EMBODIMENTS

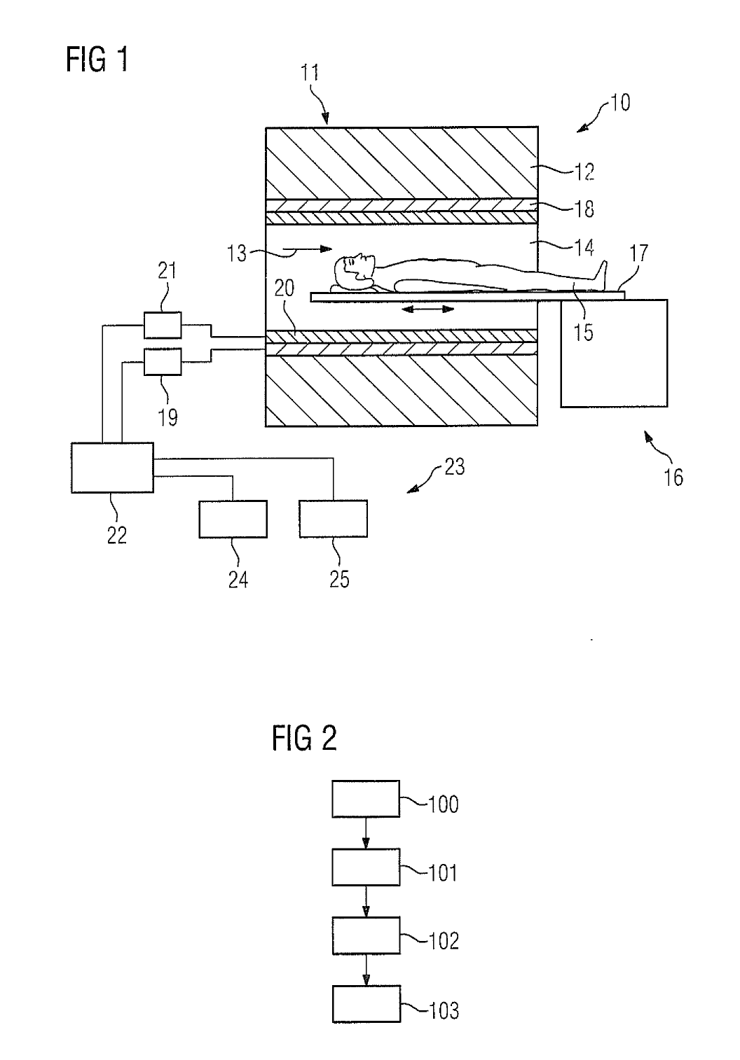

FIG. 1 schematically shows a magnetic resonance apparatus 10. The magnetic resonance apparatus 10 has a scanner 11 that has a superconducting basic field magnet 12 that generates a strong and constant basic magnetic field 13. The scanner 11 has a patient receiving area 14 for receiving a patient 15. In the exemplary embodiment, the patient receiving area 14 is cylindrical and is circumferentially enclosed by the scanner 11. In principle, embodiments of the patient receiving area 14 deviating therefrom are conceivable. The patient 15 can be moved into the patient receiving area 14 by a patient support 16. To this end, the patient support 16 has a movable patient table 17 inside the patient receiving area 14.

The scanner 11 further has a gradient coil arrangement 18 that generates magnetic field gradients for spatially encoding MR signals during imaging. The gradient coil arrangement 18 is controlled by a gradient controller 19. The scanner 11 further has a radio-frequency antenna 20 controlled by a radio-frequency antenna controller 21 so as to radiate radio-frequency magnetic resonance sequences in an examination chamber that is substantially formed by the patient receiving area 14 of the scanner 11. The radiated radio-frequency sequences give certain nuclear spins in the patient 15 a magnetization that causes those nuclear spins to deviate from the field lines of the basic magnetic field 13 by an amount known as a flip angle. As these excited nuclear spins relax and return to the steady state, they emit the aforementioned MR signals, as RF signals. Those MR signals are detected by the same antenna from which the radio-frequency sequence was radiated, or by a different antenna.

To control the basic field magnet 12, the gradient controller 19 and the radio-frequency controller 21, the magnetic resonance apparatus 10 HAS a system control computer 22. The system control computer 22 controls the magnetic resonance apparatus 10 centrally, such as for the performance of a predetermined imaging gradient echo sequence. The system control computer 22 also has an evaluation processor (not shown), for the evaluation of MR signals and/or medical image data that are acquired during the magnetic resonance examination.

In addition, the magnetic resonance apparatus 10 has a user interface 23 that is connected to the system control computer 22. Control information such as imaging parameters, and reconstructed magnetic resonance images, can be displayed on a display unit 24, for example on at least one monitor, of the user interface 23 for a medical operator. The user interface 23 also has an input unit 25 via which information and/or parameters can be entered by the medical operator during a scanning process.

FIG. 2 shows a flowchart of the method according to the invention for establishing implant information on the patient 15 used on a magnetic resonance scan with the magnetic resonance apparatus 10. The method is controlled by the control computer 22 that, for this purpose, has computer programs and/or software, which can be loaded directly into a memory of the control computer 22, with program code that cause the inventive method for establishing implant information about the patient 15 based on a magnetic resonance scan with the magnetic resonance apparatus 10, when the program code is executed in the control computer 22. The control computer 22 has a core processor configured to execute the computer programs and/or software. The code for the computer programs and/or software can also be stored on an electronically readable data storage medium embodied separately from the control computer 22, which can be loaded into the computer 22.

The magnetic resonance scan for establishing the implant information is a safety magnetic resonance scan. At the start of the method for establishing the implant information about the patient 15 based on the magnetic resonance scan, in particular the safety magnetic resonance scan, the patient 15 is positioned on the patient table 17 of the patient support 16. The method for establishing the implant information on the patient 15 with the safety magnetic resonance scan is preferably performed before a further magnetic resonance scan, in particular a medical and/or diagnostic magnetic resonance scan. In the further medical and/or diagnostic, medical image data of a region of interest are acquired. The method for establishing the implant information about the patient 15 is performed before planning the further medical and/or diagnostic, magnetic resonance scan of the region of interest of the patient 15. The method is performed automatically by the magnetic resonance apparatus, in particular controlled by the control computer 22 as long as no positive implant information on the patient are stored for the medical and/or diagnostic magnetic resonance scan.

At the start of the method, in a first method step 100, the magnetic resonance scan, in particular the safety magnetic resonance scan, is performed to establish the implant information. MR signals are acquired, and reconstructed so as to produce magnetic resonance image data. The magnetic resonance scan, in particular the safety magnetic resonance scan, is performed automatically by the scanner 11 of the magnetic resonance apparatus 10 controlled by the control computer 22. The magnetic resonance scan, in particular the safety magnetic resonance scan, is governed by implant-safe settings in the control parameters. The implant-safe settings in the control parameters preferably have a soft slew rate of gradient pulses that are activated during the safety scan.

The maximum soft slew rate of the gradient pulses is preferably 20 mT/ms at most. The maximum soft slew rate of the gradient pulses is more preferably 15 mT/ms or 10 mT/ms or 8 mT/ms or 5 mT/ms.

Alternatively or additionally, the implant-safe settings in the control parameters preferably have a maximum SAR value of almost 1.5 W/kg. The maximum SAR value is more preferably 1.4 W/kg or 1.3 W/kg or 1.2 W/kg or 1.1 W/kg or 1.0 W/kg or 0.9 W/kg or 0.8 W/kg or 0.7 W/kg or 0.6 W/kg or 0.5 W/kg or 0.4 W/kg or 0.3 W/kg or 0.2 W/kg or 0.1 W/kg. In addition, radio-frequency pulses radiated in the safety scan preferably have a maximum amplitude of 30 μT. The radio-frequency pulses more preferably have a maximum amplitude of 25 μT or 20 μT or 18 μT or 15 μT.

In addition, the implant-safe settings in the control parameters preferably designate a slice thickness of about 0.5 cm. The slice thickness may alternatively have a value of about 0.8 cm or 1.0 cm or 1.2 cm or 1.4 cm or 1.6 cm or 1.8 cm or 2.0 cm.

The magnetic resonance scan, in particular the safety magnetic resonance scan, with the implant-safe settings in the control parameters, acquires MR signals from a region of interest of the patient 15, may encompass comprises the entire body of the patient 15 so that magnetic resonance image data for the whole body of the patient 15 are available to establish the implant information.

In a subsequent method step 101, an evaluation of the reconstructed magnetic resonance image data takes place. To this end, the control computer 22 has an evaluation algorithm with which the evaluation of the magnetic resonance image data acquired is performed. During the evaluation of the magnetic resonance image data, the magnetic resonance image data is searched for weak-signal regions. The weak-signal regions preferably are signal-free regions, i.e. regions from which no MR signal was emitted in the safety scans, and are hence “seen” as black regions in the image data.

In order to identify the weak-signal regions, in particular the signal-free regions, the regions preferably have a minimum size. To this end, the aforementioned search algorithm is designed so that weak-signal regions, in particular signal-free regions, must have a minimum size of greater than 0.5 cm in order to be identified as a weak-signal regions, in particular signal-free regions. The weak-signal regions, in particular the signal-free regions, preferably must have a size of greater than 1.0 cm in order to be identified as weak-signal regions. Particularly advantageously, the weak-signal regions, have a size of greater than 1.5 cm in order to be identified as weak-signal regions.

Subsequently, in a further method step 102, implant information is generated dependent on the result of the search for the weak-signal regions, in particular signal-free regions. The generation of the implant information is preferably performed by the control computer 22 of the magnetic resonance apparatus 10.

If the image data contain at least one weak-signal region, in particular a signal-free region, the control computer 22 generates positive implant information. The positive implant information indicates that the patient 15 has at least one implant that has to be taken into account during the upcoming further medical and/or diagnostic magnetic resonance examination.

If no weak-signal region, in particular no signal-free region, is identified, the control computer 22 generates negative implant information. The negative implant information indicates that the patient 15 does not have an implant.

In this method step 102, the implant information generated is also displayed to the medical operator at the display unit 24 of the user interface 23. In addition, the implant information can be transferred to a mobile terminal, for example a smartphone or a tablet PC, belonging to a user, or to a display unit separate from the magnetic resonance apparatus 10 and displayed there. This can be beneficial, for example, if the user planning the medical and/or diagnostic magnetic resonance scan on the patient 15 is not present at the site of the magnetic resonance examination.

The generation of the implant information is followed by the performance of the further medical and/or diagnostic magnetic resonance scan for the generation and/or acquisition of medical image data of a region of interest of the body of the patient 15. This is performed in a further method step 103. In this method step 103, first the further medical and/or diagnostic magnetic resonance scan is planned and the corresponding settings, preferably determined manually by the medical operator or also automatically by the control computer 22, are made and then the further medical and/or diagnostic magnetic resonance scan is performed.

If the further method step 102 contains positive implant information, the further medical and/or diagnostic magnetic resonance scan is planned and performed in safety mode. If the further method step 102 contains negative implant information, the further medical and/or diagnostic magnetic resonance scan is planned and performed in normal mode.

Although modifications and changes may be suggested by those skilled in the art, it is the intention of the Applicant to embody within the patent warranted hereon all changes and modifications as reasonably and properly come within the scope of the Applicant's contribution to the art.

1. A method for establishing implant information in a magnetic resonance (MR) scan of a subject, said method comprising:
operating an MR data acquisition scanner to execute an MR scan with control parameters comprising implant-safe settings, while an examination subject is situated in the MR data acquisition scanner, in order to acquire MR signals from the subject;
providing the MR signals to a computer and reconstructing MR image data from the MR signals;
in said computer, evaluating said MR image data by conducting a search of said MR image data for at least one weak-signal region, and thereby obtaining a search result; and
in said computer, generating implant information about the patient dependent on said search result, and generating an electrical output signal from the computer that comprises said implant information.

2. A method as claimed in claim 1 comprising conducting said search in said computer to identify a signal-free region as said at least one weak-signal region.

3. A method as claimed in claim 1 comprising conducting said search in said computer with a requirement that any weak-signal region in said MR image data must have a minimum size of greater than 0.5 cm in order to be identified as a weak-signal region.

4. A method as claimed in claim 1 comprising operating said MR data acquisition scanner to execute said MR scan as a whole body scan of the subject.

5. A method as claimed in claim 1 comprising operating the MR data acquisition scanner with said implant-safe settings in said control parameters comprising a designation of a slice thickness, from which said MR signals are acquired, of 0.5 cm.

6. A method as claimed in claim 1 comprising operating said MR data acquisition scanner to execute said MR scan with said implant-safe settings in said control parameters comprising a soft slew rate for gradient pulses activated during said MR scan.

7. A method as claimed in claim 1 comprising operating said MR data acquisition scanner to execute said MR scan with said implant-safe settings in said control parameters requiring a maximum specific absorption rate (SAR) value of 1.5 W/kg.

8. A method as claimed in claim 1 comprising, when said search result is positive so as to indicate that an implant is present in the subject, thereafter operating said MR data acquisition scanner to execute a diagnostic magnetic resonance scan of a region of interest of the subject, with said MR data acquisition scanner operated in a safety mode.

9. A method as claimed in claim 1 comprising, when said implant information is negative, so as to indicate that no implant is present in the patient, thereafter operating the MR data acquisition apparatus to execute a diagnostic MR scan of the subject in a normal mode.

10. A method as claimed in claim 1 comprising displaying said implant information at a display monitor in communication with said computer.

11. A magnetic resonance (MR) apparatus comprising:
an MR data acquisition scanner;
a computer configured to operate the MR data acquisition scanner to execute an MR scan with control parameters comprising implant-safe settings, while an examination subject is situated in the MR data acquisition scanner, in order to acquire MR signals from the subject;
said computer being configured to reconstruct MR image data from the MR signals;
said computer being configured to evaluate said MR image data by conducting a search of said MR image data for at least one weak-signal region, and thereby obtaining a search result; and
said computer being configured to generate implant information about the patient dependent on said search result, and generating an electrical output signal from the computer that comprises said implant information.

12. A non-transitory, computer-readable data storage medium encoded with programming instructions, said storage medium being loaded into a computer system of a magnetic resonance (MR) apparatus comprising an MR data acquisition scanner, said programming instructions causing said computer system to:
said MR data acquisition scanner to execute an MR scan with control parameters comprising implant-safe settings, while an examination subject is situated in the MR data acquisition scanner, in order to acquire MR signals from the subject;
reconstruct MR image data from the MR signals;
evaluate said MR image data by conducting a search of said MR image data for at least one weak-signal region, and thereby obtaining a search result; and
generate implant information about the patient dependent on said search result, and generate an electrical output signal from the computer system that comprises said implant information.
